(19) United States
(12) Patent Application Publication
(10) Pub. No.: US 2025/0002895 A1
(43) Pub. Date: Jan. 2, 2025

(54) ELECTROPORATION APPARATUSES AND THEIR METHOD OF USE

(71) Applicants: Emily Chen Tang, Warren, NJ (US); Bryan Michael Gerber, Warren, NJ (US); Chadwick Phillip Pearson, Warren, NJ (US); Harsimran Panesar, Stirling, NJ (US)

(72) Inventors: Emily Chen Tang, Warren, NJ (US); Bryan Michael Gerber, Warren, NJ (US); Chadwick Phillip Pearson, Warren, NJ (US); Harsimran Panesar, Stirling, NJ (US)

(21) Appl. No.: 18/885,920

(22) Filed: Sep. 16, 2024

Related U.S. Application Data
(63) Continuation-in-part of application No. 17/579,701, filed on Jan. 20, 2022, now Pat. No. 12,091,654.
Application No. 17/579,701 is a division of application No. 14/679,327, filed on Apr. 6, 2015, now Pat. No. 11,260,137.
(60) Provisional application No. 61/975,828, filed on Apr. 6, 2014.

(51) Int. Cl. C12N 13/00; A23L 3/32; A61L 2/08; B08B 6/00

(52) U.S. Cl. CPC C12N 13/00; A23L 3/32; A61L 2/08; B08B 6/00

(57) ABSTRACT

The device uses pulsed electric fields to prevent the growth of biofilm and the attachment of bacteria to targeted surfaces. The device sets up an electric field around or surrounding the surface itself. These pulsed electric fields disrupt biofilm formation and bacterial attachment to surfaces. The device is meant to prevent the formation of biofilm or attachment of bacteria to a surface as opposed to disinfecting the surface. The device is able and ideally suited to be used for a plurality of purposes including but not limited to being used with beer lines to taps, ice makers, chillers or other HVAC systems, and containers for storing medical equipment such as dentures or other medical devices that may be stored in water.

This application claims priority under 35 USC 119(e) and 35 USC 120 and is a continuation-in-part of U.S. Non-Provisional application Ser. No. 17/579,701 filed Jan. 20, 2022, which is a divisional application of U.S. Non-Provisional application Ser. No. 14/679,327 filed Apr. 6, 2015, which claims priority to Provisional Patent Application No. 61/975,828 filed on Apr. 6, 2014, the contents of all of which are incorporated by reference herein in their entireties.

### FIELD OF THE EMBODIMENTS

The present invention relates to the use of pulsed electric fields. In particular, the present invention relates to using pulsed electric fields to cause electroporation in various biofilms and/or prevent surface bacterial contamination.

### BACKGROUND OF THE EMBODIMENTS

Biofilm is a collection of microbial cells that are tightly bound a matrix of primarily polysaccharide material. These biofilms frequently exhibit strong bonding properties, and are incapable of being removed through gentle rinsing. One common problem in industries such as food processing is the buildup of biofilm and attachment of organisms to certain surfaces. For example, this buildup frequently occurs within the various tubes used throughout the food processing industry.

The prior art contemplates various methods for removing biofilm. However, many of the standard methods used in many food-processing industries today, such as alkali-based as well as acid-based cleaners, are capable of adequately removing extracellular polymeric biofilm matrix only when the correct process parameters (e.g. formulations, concentrations, time, temperature) are applied. The removal of biofilm and its attached bacteria can be facilitated through the use of mechanical force to the surface during cleaning. Common examples of these applications of mechanical force are brushing and scrubbing. The mechanical removal of biofilm and attached bacteria can be achieved via high-pressure spray hoses, however, these hoses cause the dispersion of bacteria over a wide area, which often results in the growth of mold and bacterial. Further, the interior of certain surfaces, such as tubing, is not readily accessible to be cleaned via mechanical force. Note that all of the above-mentioned techniques are all methods of treatment, as opposed to methods of prevention.

In contrast, the present invention employs the use of Pulsed Electric Fields (hereinafter “PEF”). PEF's are high intensity electric fields which are capable of disrupting a number of bacterial functions. Generally, PEF's are created by a power supply creating a difference in the electrical potential between two electrodes. PEF's are used to prevent the formation of biofilms because are capable of causing electroporation in a wide variety of bacteria. Electroporation is the process of PEF's (or some other kind of electric field) causing small holes to form in the membrane of a given bacterium. Electroporation leads to decreased cell viability and proliferation in many different bacteria.

One beneficial use of PEF's is in the food processing industry. There, PEF's are often used as an alternative to other pasteurization methods, due to the minimal heat generated when exposing a food stuff to a PEF. This has the benefit of limiting thermal degradation, a serious problem in the food processing industry. The prior art contains a few systems which do use PEF, albeit as a disinfectant. However, these systems must employ the use of a small chamber due to the fact that electric field is reduced as the distance between electrodes is increased. Further, these systems are difficult to scale up, and frequently must employ a vacuum system to prevent air bubbles from causing a fire hazard.

Another beneficial use of PEF's is in the remove dental plaque. Previously a number of different devices have been used to remove dental plaque. A review of the previous solutions to removing dental plaque is provided:

Toothbrush: Over the years the brush head shape, filament shape and filament insertion method into the handle have changed in order to be most effective and comfortable. However, a detriment of all existing toothbrushes is that they need to be replaced every 3 or 4 months to be effective at cleaning one's mouth. Further, even when the brush head is appropriately maintained, according to P&G Oral Health, only a mere 65.1% of the plaque is removed by a manual brush.

Electric toothbrush: Electric toothbrushes are widely used because of their convenience, but the electric motors that power them may produce electromagnetic waves. It has been shown that electric toothbrushes generate low frequency magnetic fields (1-2000 Hz) which induce an electric current in dental appliances such as orthodontic and prosthetic appliances and dental implants. Further, accordingly to P&G Oral Health, only 73.6% of the plaque is removed by an electrical toothbrush.

Ultrasonic toothbrush: Device used to remove plaque, from teeth. Comprises of a toothbrush having a hollow, cylindrical handle that holds an electrical motor and is power by batteries (DC). The electrical motor (transducer) generates high frequency (above 20 kHz) mechanical waves along the brush. The ideal frequency of the ultrasonic pressure waves is between 0.75 MHZ and 1.6 MHZ, but it can range from 20 kHz to above 2000 kHz depending on the selection of materials utilized in the toothbrush and the components in the dentifrice to maximize cavitation and acoustic streaming in the fluids of the oral cavity. Maximum level of ultrasound energy coupled to the fluids within the oral cavity and to the teeth and gums and periodontal pockets, achieving maximum loosening of soft plaque. This technology has also been applied to dental scalar, however because of the high energy flow the scalars (and the toothbrushes) can heat up. Such devices can be uncomfortable because of the high intensity vibrations and high pitched audible sounds. Therefore, these devices had been limited to the dentist's office. But recently, a new design of the ultrasonic brush creates standing sound waves allows the device to be used at home. The time averaged intensity of the ultrasonic pressure waves is now ideally limited to approximately 30 mW/cm², which is effective for the purpose and at the same time it is below the tissue heating range. However, higher intensities can be applied with the appropriate safeguards against tissue heating or damage. The ultrasonic pressure waves could be applied in a continuous wave modality or in a pulsed burse mode modality such as 200-microsecond burse width repeated at 1 KHz repetition rate to further limit tissue heating.

Dental floss: To remove interproximal plaque, dental floss is a very popular tool. However, achieving proper flossing technique can be difficult, resulting in this technique being less than universal in its application. Despite this. The American Dental Association (ADA) even reports that up to 80% of plaque may be removed by this method. However, a study aimed to assess systematically the effect of the use of dental flood in addition to the use of a tooth brush, as opposed to the effect of the use of a toothbrush alone on interproximal plaque and gingivitis, found that the use of dental floss provides no benefit. A subsequent study found that flossing was only effective in reducing interproximal caries risk when applied professionally. Their systematic review showed that high-quality professional flossing performed in first grade children on school days reduced caries risk by 40%. Self-flossing, on the contrary, failed to have the same beneficial effect.

Dental Scaler: A dental scaler is a hand-held device that has a metallic end shaped like a hook or curved blade and can be ultrasonic. To use the scaler, a user scrapes the long edge of the scaler's blade along each tooth's surface, from just above or below the gum line toward the end of the tooth. However, this is not something that should be done daily, and do properly wield such a scaler requires caution, patience, and skill. Further, scraping too roughly can damage the enamel on the teeth (which does not regenerate), scratch implants, as well as cut a patient's gums. All of these scratches lead to a patient's teeth permanently having an increased surface area; giving bacteria a wider array of surfaces to grow on.

As has been shown above, the solutions in the prior art have a number of inadequacies with regard to the effectiveness of the treatment. None of the art described above addresses all of the issues that the present invention does.

### SUMMARY OF THE EMBODIMENTS

The present invention provides for an electric field generator, comprising: a power source, having an output; a first electrode connected to said output of said power source; a second electrode, wherein said second electrode is grounded, wherein said first electrode and said second electrode are situated such that an electric field is generated in the space between said first electrode and said second electrode, and wherein said electric field has an intensity ranging from 5 V/cm to 2000 V/cm. In a preferred embodiment, this electrical device generates an electric field with an intensity ranging from 5 V/cm and 2000 V/cm, preferably 1000 V/cm to 2000 V/cm. In alternative embodiments, this electrical device is incorporated into tooth brushes, dental trays, and soda dispenser machines. The present invention also contemplates a method of preventing or reducing biofilms on a substrate, comprising the steps of: coating, said substrate with a conductive polymer; generating, a pulsed electric field with an intensity of at least 400 V/cm; exposing, for a period of at least five minutes, said substrate to said electric field.

The present invention also contemplates a method of causing electroporation in at least one cell, comprising: generating, an electric field from a power source generating alternating current operatively attached to a plurality of electrodes; exposing, said at least one cell to said electric field. These cells can be that of bacteria, or that of plant cells. Electroporation of plant cells can the ability to extract oil and lipids from small aquatic plants.

The current invention uses Pulsed Electric Fields (PEF) technology to prevent biofilm growth and bacterial attachment to surfaces. PEF are used to disrupt cell communication and cell attachment and therefore disrupt bacterial attachment and biofilm formation. This is different from the current use of high intensity PEF for disinfection. This invention uses lower intensity electric fields and less power than required for disinfection, to prevent bacterial attachment to surfaces. The electric fields used in this invention have the strength in the range of 2-20 kV/cm and a medium frequency range of 5-50 kHz. The application of PEF in this invention involves the use of fully insulated electrodes, which can be applied externally to devices and surfaces, ex. inside surfaces of closed tubing in food processing plants, implants, dental implants, or teeth. The high voltage insulation around the electrodes prevents current (except a minimal, <microAmp range leakage current) resulting in low power use. The mid-frequency range results in low radiation emission. As a result the invention can be scaled up to a large size without large power inputs. It can be used with existing equipment or built-in during manufacturing and easily incorporated into existing systems.

The prevention of biofilm formation saves the time spent to clean surfaces. Since the aim is prevention, as opposed to disinfection, many of the limitations of previous PEF systems do not apply. The present invention can be made to any size considering the dimensions of the surface [that has the PEF applied to it] can be varied. Therefore our invention can be easily incorporated into existing systems. Referring back to the sample application in the food industry, some of the limitations are avoided. Where there previously was a small PEF treatment chamber set-up (which was previously needed to minimize electrode separation distance) can be eliminated. The electrodes in the present invention can be further apart due to the fact that weaker electric fields are needed. Also due to the larger electrode separation distance the presence of bubbles is not as detrimental. These are just sample limitations that can be avoided if the present invention was applied in one of its possible fields, the food industry.

Regarding PEF's use in removing dental plaque, the electric field changes the bacteria formation and thus naturally decreases the bacteria growth rate. This gentle cleaning approach reaches places that you cannot reach and ensures the whole mouth gets cleaned. The present invention is used just like a normal toothbrush along with your favorite toothpaste but has longer effect on the plaque. The present invention does not take place of your daily oral hygiene routine. You still need to brush and floss regularly, but the present invention enhances your daily cleaning. The present invention does not depend on current to affect the bacteria, since the current out is minimal, close to zero. Rather, the present invention uses the electric field which uses voltage and high frequency to out force on the bacteria changes and disrupting its physiology.

Also, due to the ability of PEF's to remove dental plaque, the present invention is of particular use for cleaning dentures. PEF's are suitable for the mass sterilization of pre-packaged foods, providing a fast, efficient, and cheap means to ensure the sterilization of processed foods.

### BRIEF DESCRIPTION OF THE DRAWINGS

FIG. 1 is a front perspective view of an embodiment of the present invention.

FIG. 2 is a side view of an embodiment of the present invention.

FIG. 3 is an alternative side view of an embodiment of the present invention.

FIG. 4 is a side view of an alternative embodiment of the present invention.

FIG. 5 is a side view of another embodiment of the present invention.

FIG. 6 is a side view of a cross-section of another embodiment of the present invention.

FIG. 7 is a perspective view shows a possible surface to high voltage connector layout.

FIG. 8 is a perspective view of a possible surface layout of an embodiment of the present invention.

FIG. 9 is a side view of an electric field treatment chamber.

FIG. 10 is a perspective view of a single electrode embodiment of the present invention.

FIG. 11 is a perspective view of a possible electrode wrap design.

FIG. 12 shows a perspective view of a system with beer kegs and beer lines leading to taps using the device of the present invention.

FIG. 13 shows a close-up perspective view of the beer lines leading to taps using the device of the present invention.

FIG. 14 shows a perspective view of a system with soda and/or beer lines leading to soda fountain dispensers using the device of the present invention.

FIG. 15 shows a schematic view of a icemaker system using the device of the present invention.

FIG. 16 shows an advantage of the device of the present invention with optimal heat exchange efficiency (for example, in an HVAC system).

FIG. 17 shows an exemplary cooling tower system using the device of the present invention.

FIG. 18 shows a perspective view of a heat exchanger using the device of the present invention.

FIG. 19 shows a perspective view of a storage container for dentures or false teeth using the device of the present invention.

### DESCRIPTION OF THE PREFERRED EMBODIMENTS

The preferred embodiments of the present invention will now be described with reference to the drawings. Identical elements in the various figures are identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

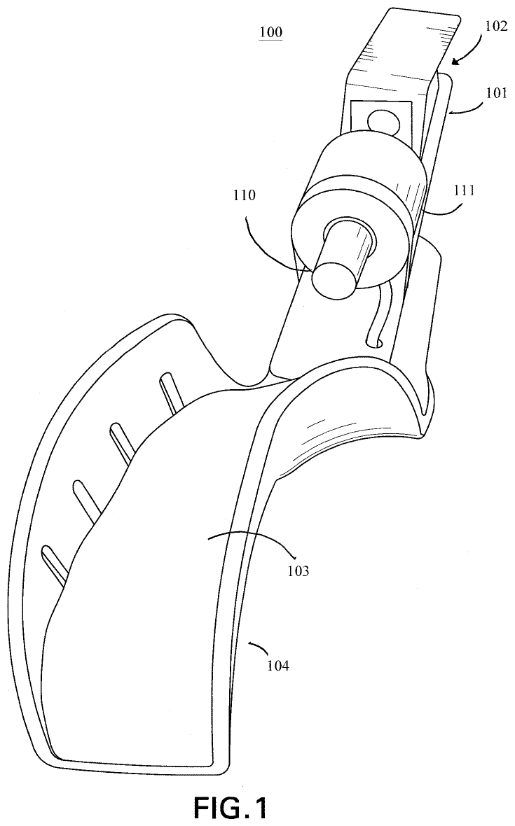

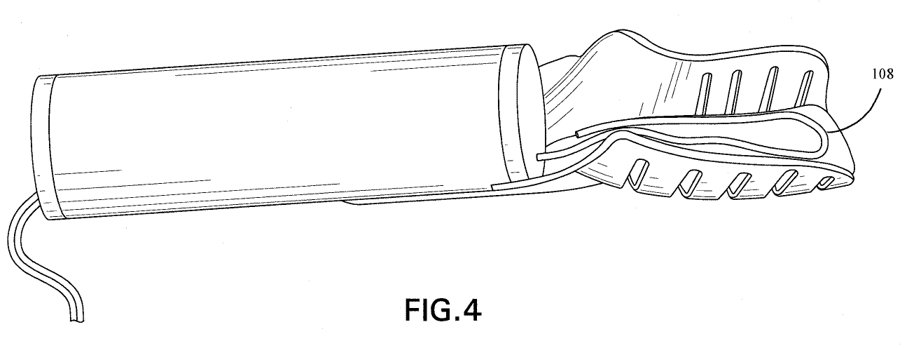

Referring to FIG. 1, a front perspective view of an embodiment of the present invention is provided for. Here, electrical device 100 is incorporated into a dental tray. This embodiment is comprised of power supply 101, insulation 103, dental tray 104, first insulation 111, first electrode 110, and second electrode 108 (See FIG. 4). This embodiment may be used to clean the inside of a user's mouth when a user inserts dental tray 104 into their mouth and activates electrical device 100. This embodiment may also be used to clean dentures or other artificial teeth by resting those products on dental tray 104 and activating electrical device 100 via power supply 101. Also equipped in this embodiment is AC adapter 102. In a preferred embodiment, power supply 101 is located in close proximity to AC adapter 102. In another preferred embodiment, power supply 101 and AC adapter 102 are one and the same. Insulation 103 is used to cover up electrode 108. Note that due to the nature of pulsed electric fields, as well as the nature of this particular embodiment, electrode 108 must be completed insulated. This provides for safety for the user as well as increased efficiency in generating said pulsed electric field. In this regard, insulation 103 may be comprised of a single piece that envelops electrode 108 or may be comprised of two pieces; one on top of electrode 108 and one below electrode 108, forming a seal where the two pieces meet.

While this particular embodiment of electrical device 100 is equipped with AC adapter 102, this disclosure contemplates an embodiment that is powered solely by DC power. In that embodiment, there is no need for AC adapter 102.

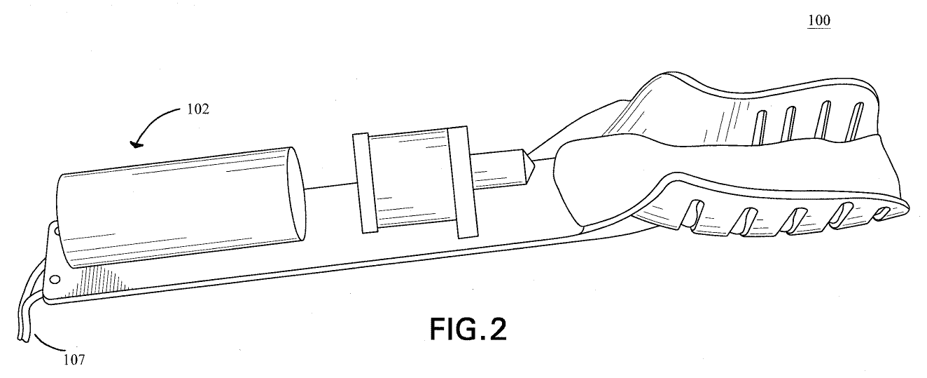

Turning to FIG. 2, is a side view of an embodiment of the present invention is provided for. While this embodiment is substantially similar to that embodiment shown in FIG. 1, this embodiment is equipped with the optional AC adapter output 107. When electrical device 100 is equipped with AC adapter 102, it is preferably equipped with AC adapter output 107. Note that, as previously mentioned, electrical device 100 may be operated solely via direct current, but it may also be operated with alternating current as well, if equipped with AC adapter 102 and AC adapter output 107.

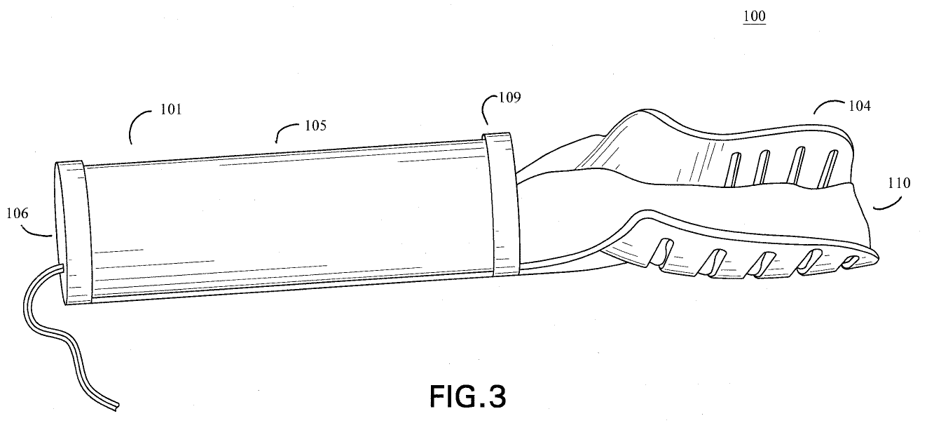

FIG. 3 shows an alternative side view of an embodiment of the present invention. Here, electrical device 100 is still equipped with power supply 101, first electrode 110, and first insulation 111 (See FIG. 1), optionally equipped AC adapter 102 and AC adapter output 107 (See FIG. 2), second insulation 103, and dental tray 104. In addition to those components, this view shows insulated lid 106, insulated cover 105, and lid 109. These additional components provide for complete insulation of power supply 101 and first electrode 110, allowing for greater user safety. Note the distinction between insulated lid 106 and lid 109. Lid 109 cannot be insulated, otherwise the PEF would not be generated. In contrast insulated lid 106 is insulated so that a user's hand is not subjected to the PEF.

Referring to FIG. 4, is a side view of an alternative embodiment of the present invention is provided for. Here, second electrode 108 is shown by removing the top layer of second insulation. By placing second electrode 108 on top of the dental tray, a PEF will be generated around the area of a user's teeth while using the device.

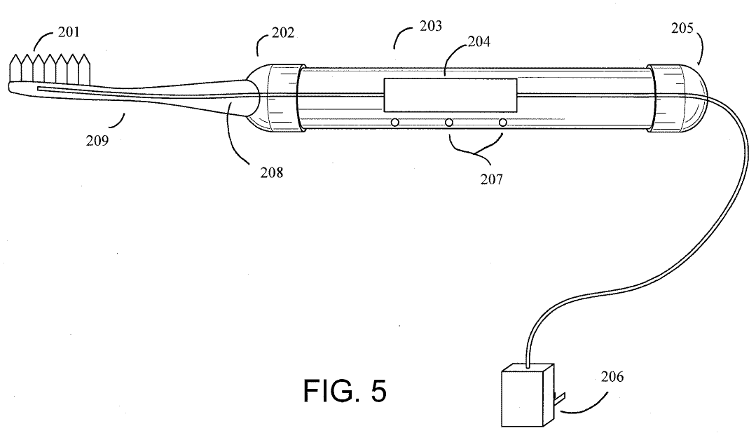

FIG. 5 is a side view of another embodiment of the present invention.

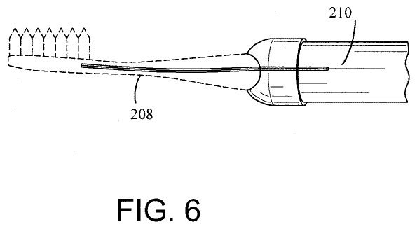

FIG. 6 is a side view of a cross-section of another embodiment of the present invention. [MM]

This invention involves the application of electric field to a region above and/or surrounding a surface with the aim of preventing bacteria from attaching to the surface. A general design includes one or two high voltage electrodes thoroughly insulated with high voltage insulation such as glass, ceramic, or plastic insulation to prevent any sparking or current through the system. The specific electrode geometry depends on the specific application and several examples are given here.

In an alternative embodiment of the present invention, electrical device 100 can be used in the processing of certain liquids such as juices or dairy products. The present invention has a variety of embodiment, each made at different sizes. This allows the present invention to be easily incorporated into any pre-existing manufacturing system. For example, a given food processing plant would merely have to replace their tubes with this embodiment of present invention.

It may be used in other situations where preventing bacterial attachment to a surface is warranted.

In various embodiments, the present invention is not only applicable to permanently fixed object, but may be used to subject bacteria on a number of different surfaces to a PEF.

Preferably, the pulsing parameters for power supply 101 are between 0.1-20 kV/cm with a frequency range of 1 μHz to 100 MHz or between 2 and 20 kV/cm and between 5 kHz and 60 KHz. This provides for electric fields that are strong enough to disrupt the cells, but are not as high as those used for disinfection. A power source and the frequency ranges is chosen to reduce the cost of the device because the power sources cost less and to make the devices is safe for public use. In a preferred embodiment, the pulsed electric field is not applied continuously. Rather, the power source is on for a short time and off for a much longer time, therefore reducing the duty cycle to increase efficiency. The choice of a specific duty cycle is dependent on the doubling time for the bacterial species targeted.

The device outlined is innovative in that it uses pulsed electric fields as a way to prevent biofilm growth and bacterial attachment as opposed to disinfection.

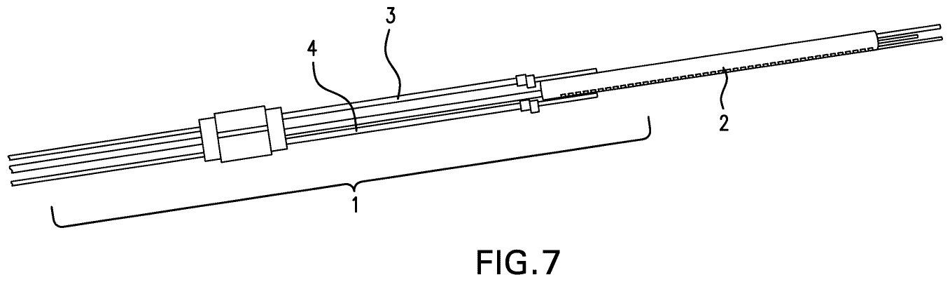

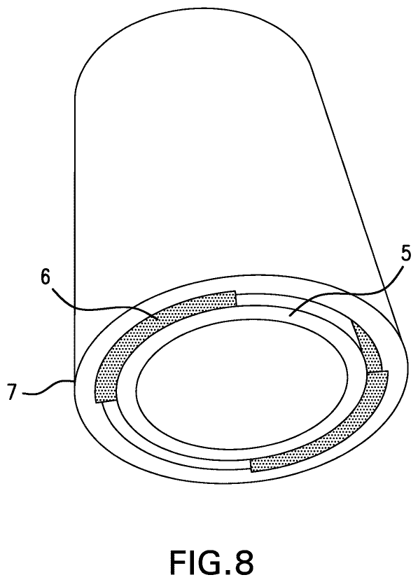

Referring to FIG. 7 and FIG. 8, tubing 2 is used. Surfaces, an example of such is tubing 5, will have two electrodes 6 applied to the surface 5. The electrodes 6 are attached to a high voltage connector 1; one of the electrodes is attached to the high voltage wire 3 from the connector and the other electrode is attached to the neutral wire from the connector 4. This connector will be attached into a high voltage high frequency power supply. All electrodes 6 are insulated 7.

A possible use of the sample invention in FIG. 7 is in the processing of certain liquids such as juices or dairy products. The invention can be made at variable sizes, so it can be easily incorporated into any pre-existing manufacturing system. A processing plant would simply have to replace their tubes with this present invention, provide an electrical source for the power supply, and then let the system run. Although the preceding description of the present invention contains significant details, it should not be construed as the limiting scope of the invention but rather as possible application of the invention. It may be used in other situations where preventing bacterial attachment to a surface is warranted.

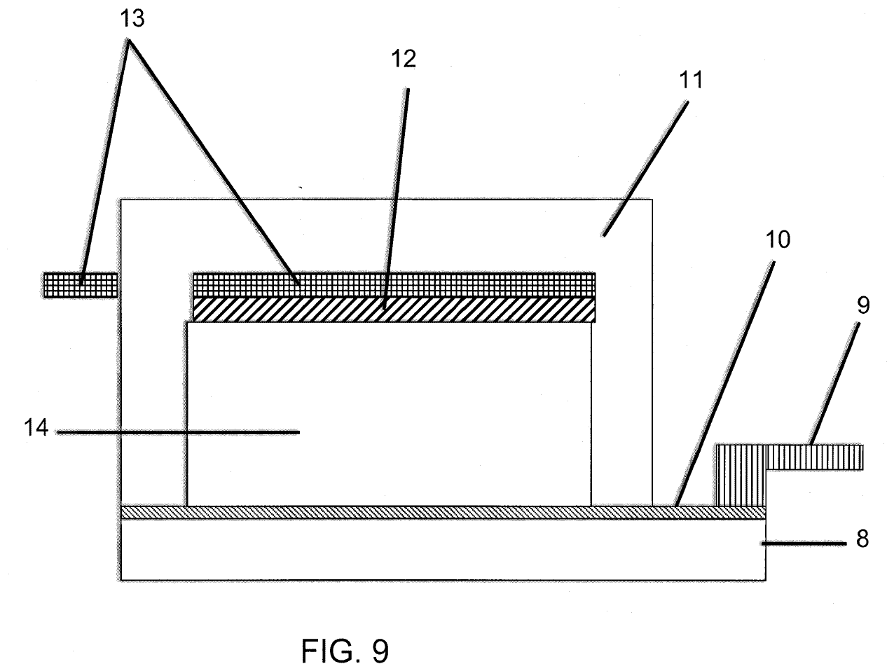

The invention is not only applicable to permanently fixed object. Surface disinfection can apply to many surfaces. FIG. 9 shows another method of applying the electric fields used for prevention of biofilm growth and/or bacterial contamination. An insulating base 8 and structure 11 can be used to hold electrodes. An electrode 10 is grounded 9 while another electrode 13 has the high voltage applied to it. This high voltage electrode 13 is insulated 12. The space between the two electrodes is the “treatment chamber” 14. This set-up FIG. 9 is applicable to, but not limited to, such objects as dentures. Continuing with the example of dentures, when they are not in use they can have biofilm growing or bacterial infecting them; this is when the present invention will be used to prevent this bacterial growth.

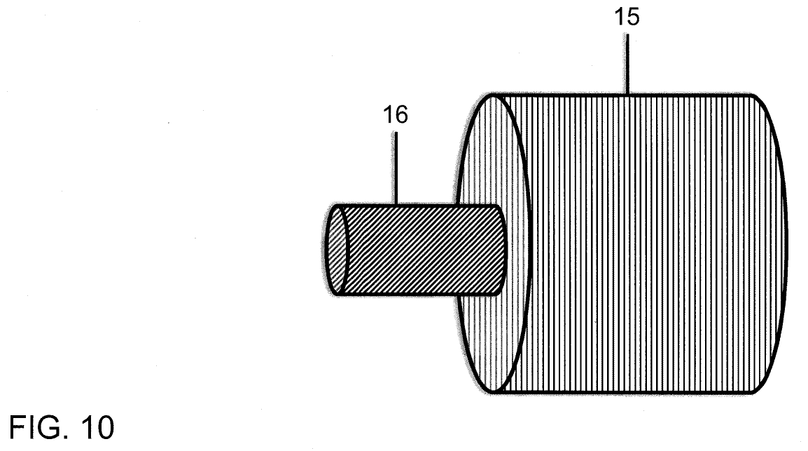

FIG. 10 shows a possible single electrode design. A high voltage electrode 16 is insulated 15 to prevent current leakage and to ensure safety of the product. Examples of applications of such as design include but are not limited to electric field treatment of teeth, medical tools, and implants.

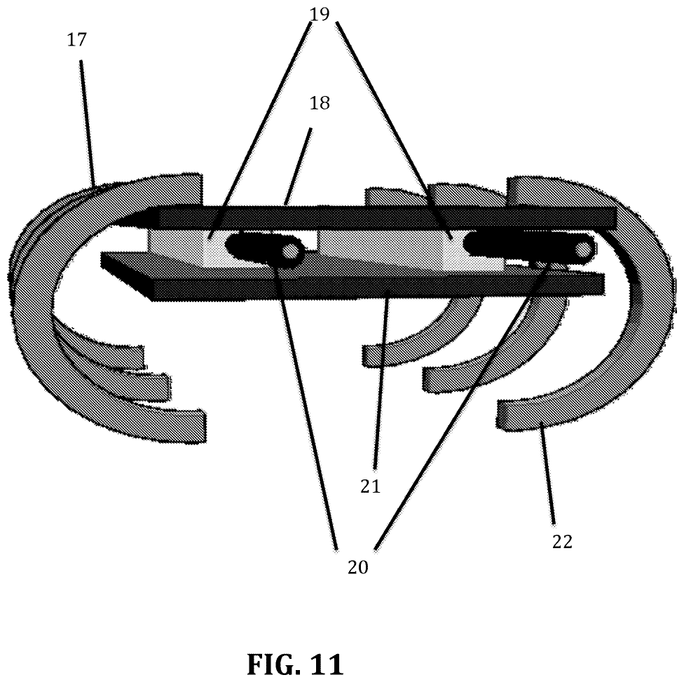

Referring to FIG. 11, left wrap adhesive 17 attaches to right adhesive 22 when inner insulation 21 is curled around a given surface 5. A plurality of adhesives 17 and 22 can be used to secure one end of the wrap around the tube to another end. One of either the left wrap adhesive 17 or right wrap adhesive 22 can be eliminated if one is sufficient to attach the wrap design to a surface. Any number of electrodes 20 can be insulated by any number of insulators 19. These insulated electrodes may be positioned between the inner wrap 21 and outer wrap 18. In some instances, the inner wrap 21 and outer wrap 18 may provide insulative properties thereby removing the need for the insulators 19.

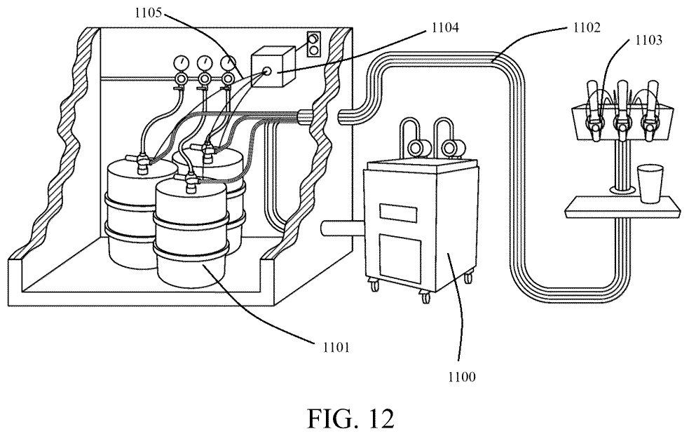

FIG. 12 to FIG. 19 show a plurality of situations in which the device of the present invention can be used. For example, the device of the present invention can be used with delivery systems for draft beer, with HVAC systems that use water, with ice makers, with soda fountain dispensers, and with containers in which medical equipment is stored in water.

FIG. 12 shows a perspective view of a system with beer kegs and beer lines leading to taps using the device of the present invention. The system known as a beer trunkline comprises a compressor or chiller 1100, beer kegs 1101, beer lines 1102, and beer dispensers/taps 1103, the pulse generator 1104 with wires 1105 coming from the pulse generator and going to the beer kegs, continuing along the beer lines 1102 and eventually to the beer dispensers/taps 1103. The pulsed electric field can be used as described herein, which should prevent the development of biofilms and bacterial growth in the beer kegs 1101, beer lines 1102, and beer dispensers/taps 1103.

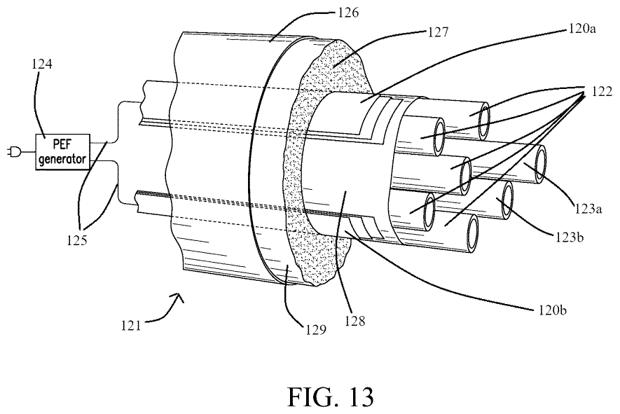

FIG. 13 shows a close up view of a beer line 121. Electrodes 120a and 120b are insulated and have three layers associated with them (with the middle layer being the electrode and the outer layers being the insulation) and are able to have a pulse applied to them by the PEF generator 124 through wires 125, which will preclude the growth of biofilms and bacteria in the beer line 121. In the beer line, one has the various beer hoses 122 that allows the passage of the beer from the beer kegs (not shown) to the beer dispensers/taps (also not shown but both are shown in FIG. 12). Cooling hose 123a allows passage of cold water or a cold water/glycol mix (such as propylene glycol) that allows for the cooling of the various beer hoses 122. It should be noted that hose 123b is also a hose that may cool the beer lines but may not be as cool as cooling hose 123a because it is the return hose that is returning the cooling mixture to the compressor or chiller (best seen in FIG. 12). That is, the cooling hose 123a has just come from the chiller or compressor and the hose 123b is returning the cooling mixture back to the compressor or chiller where the cooling mixture is cooled by the chiller or compressor. PVC sheathing 126 holds the beer line 121 together. Outer foil wrap 129 and inner foil wrap 128 bracket insulation 127. The outer foil wrap 129 and inner foil wrap 128 have as their principal purpose keeping the various beer various beer hoses together. The insulation 127 insulates the various beer hoses 122 and the cooling hose 123a and hose 123b allows for the various beer hoses 122 to remain cold. When a pulse is applied by the PEF generator 124, the electrodes 120a and 120b have the effect of precluding formation of biofilms and/or the build-up of bacteria in the various beer hoses 122 and also in the cooling hose 123a and the hose 123b. It should be noted that in an embodiment the electrodes 120a and 120b go the full length of the beer line 121 so that the pulse is applied throughout the full length of beer from the beer kegs to the beer dispensers/taps. The electric field is understood to also impact the various fittings included in the draft system.

Generally and traditionally, beer lines are cleaned by treatment with an alkaline solution and the use of the device as described herein greatly reduces the need for using the often caustic alkaline solutions that are traditionally used to get rid of biofilms and bacteria. In a variation, the use of the device as described herein will mean that a beer trunkline owner will need to perform fewer cleanings using alkaline solution. That is, the time between cleanings will be increased because bacterial/biofilm formation has been reduced. If the time between cleanings is kept at a constant interval, reduced biofilm formation in the same amount of time can possibly lead to safer drinking conditions or less biotic impact on the flavor of the beer. Moreover, when the alkaline solution is used in combination with the device of the present invention, better results are/should be attained. PEF increases the effectiveness of antibiotics (and even antibacterial properties of beer hops), so running the electric field while cleaning may lead to a deeper clean by a synergistic effect of facilitating the chemicals to penetrate microorganisms. It should be noted that when the caustic solution used to clean beer lines is not completely evacuated after cleaning, the caustic solution has been known to make people ill or even kill them. Thus, because the frequency of and the need for using alkaline solution should be reduced by use of the device as described herein, the potential danger to consumers will also necessarily be reduced.

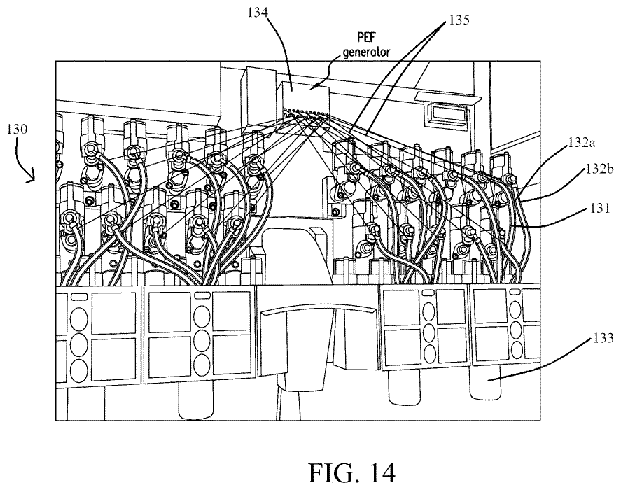

FIG. 14 shows a perspective view of a soda fountain dispensing machine with the device of the present invention. The fountain dispensing machine 130 comprises a PEF generator 134, and wires 135 coming from the PEF generator 134 and going to the soda dispensing hoses 131. On the soda dispensing hoses 131 are electrodes 132a and 132b. The PEF generator 134 generates a pulse, which is sent to the electrodes 132a and 132b, which results in the destruction of biofilms and/or bacteria in the soda dispensing hoses 131. The fountain soda is dispensed through the dispensing chute 133. Chute 133, optionally has one or more electrodes associated with the chute 133.

It should be understood that in beer line situations there are plurality of metal components such as fittings, couplers, keg connectors and disconnects, beer tower materials, beer faucet, shanks, regulators, and clean-in-place caps that are within or near the electric fields. It should be understood that these components can be intentionally designed to use materials known to have low response to electric fields. Moreover, although the metal components are discussed with reference to beer lines, materials that are used for any of the embodiments disclosed herein should also be selected so as to have a minimal response to electric fields. For example, for the soda dispenser discussed in relation to FIG. 14 should have their materials intentionally designed with electric field response in mind. Accordingly, examples of materials that can be used include but are not limited to ceramics, glass, rubber, mica, silicon carbide, plastics, polyethylene, high-density polyethylene, low density polyethylene, polypropylene, polystyrene, polyvinyl chloride, Teflon (polytetrafluoroethylene), epoxy resins, vinyl, polycarbonate, and/or nylon, or combinations thereof.

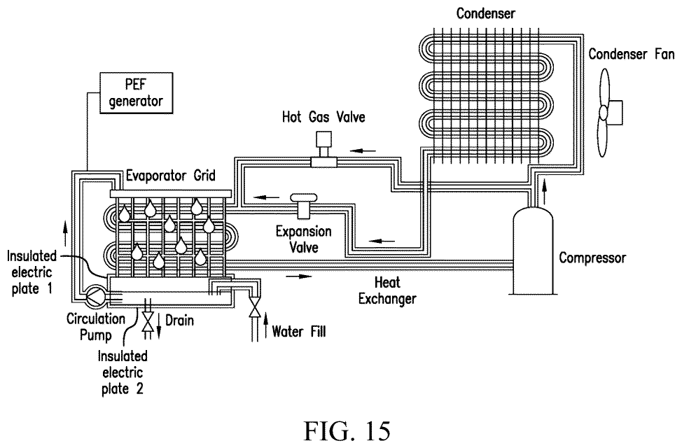

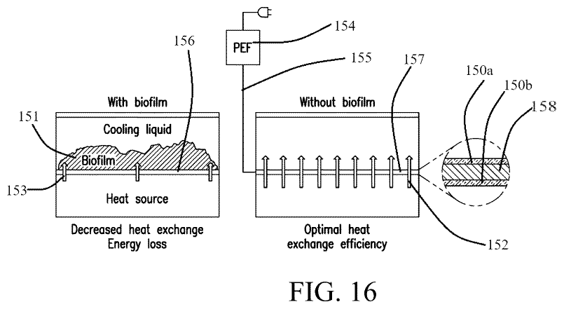

FIG. 15 shows a schematic view of an ice machine using the device of the present invention. It should be noted that the principal behind an ice machine is very similar to that used for HVAC systems. That is most of the requisite parts of an HVAC system such as a compressor, condenser coils, evaporator coils, expansion valves, condenser fan(s) are present in an ice maker. The PEF generator generates a pulse that is sent to a position that is close to the evaporator grid where there are electrodes (i.e., insulated electric plates 1 and 2 in FIG. 15) are positioned so as to prevent the formation of biofilms and/or buildup of bacteria in an area that the water is introduced into the ice maker by the water fill. The water is sent via a circulation pump to the evaporated grid where the ice cubes are made. Refrigerant in the lines of the ice maker enter the evaporator grid as a cold gas/liquid after encountering the expansion valve. This results in an evaporator grid that is sufficiently cold so as to generate ice cubes (i.e., less than 0° C. at 1 atmosphere atmospheric pressure). The refrigerant circulates eventually to the compressor wherein the refrigerant is compressed prior to being sent to the condenser and a fan, which has the effect of removing heat from the refrigerant. The refrigerant with its heat removed eventually recirculates to the evaporator grid as a cold gas/liquid to continue making ice cubes. Although insulated plates are shown positioned around an area where the water is introduced into the ice machine prior to encountering the circulation pump, it should be understood that the plates may be present after the circulation pump or alternatively, both before and after the circulation pump (including in the evaporator grid). FIG. 16 shows the difference in a heat exchanger between a heat exchanger that does not have the device of the present invention (shown on left) and a heat exchanger that employs the device of the present invention (shown on right). The presence of biofilm 151 in the heat exchanger without the device of the present invention leads to inefficient heat exchange between a heat source and a cooling liquid because the biofilm prevents efficient heat transfer through barrier 156. Arrow 153 and its size (which is smaller than arrow 152) show that there is an inefficient heat exchange occurring meaning that there is a concomitant loss of energy. For example, if the heat exchanger is present on an HVAC system (e.g., on a chiller), energy is lost during the heat exchange leading to inefficient heat exchange, which means that the chiller does not work as well. This results in increased costs and/or poorly cooled buildings. In contrast, the heat exchanger with the device of the present invention demonstrates optimal heat exchange efficiency (because no biofilm is present). In the barrier, there is insulation 150a and 150b and a single electric plate 158 that is designed to receive an electrical pulse from the PEF generator 154 through wire 155 in barrier 157 thereby preventing the formation of biofilm and/or the buildup of bacteria. Arrows 152 demonstrate that heat exchange is much more efficient through barrier 157 in the heat exchanger that has the device of the present invention relative to the heat exchanger without the device.

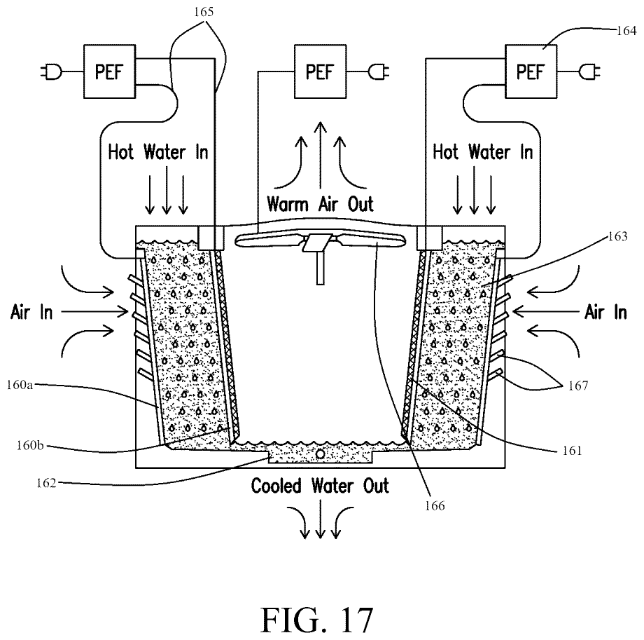

FIG. 17 shows a schematic view of a cooling tower. A cooling tower is a heat exchanger that uses air and water to reduce the temperature of water. Cooling towers are often used to cool water from air conditioners, chillers, and other industrial processes that generate heat. A cooling tower works by bringing air and water together in direct contact with each other in order to reduce the water's temperature. As this occurs, a small volume of water is evaporated, reducing the temperature of the water being circulated through the tower. As shown in FIG. 17, hot water enters the water tower at the top of the tower (usually from a pump that pumps the water to the top). The water enters the water tower and cascades through fills 163. Air passes in through louvres 167, which has the effect aiding in the cooling of the cascading water as it passes through the fills 163 (which are generally made of polymers or wood). Drift eliminator 161 are designed in a way such as to capture large water droplets caught in the cooling tower air stream. The cascading water collects in basin 162 as cool water wherein it can be used through the building to aid in cooling. The PEF generator(s) 164 generate(s) (a) pulse that is sent through wires 165 to one or more electrodes/plates 160a and 160b, thereby preventing the formation of biofilm or the buildup of bacteria in the cascading water. Fan 166 (which may be a lumen fan or any other type of fan) aids in expelling moist warm air out to the atmosphere aiding in cooling. In a variation, there may be present plates above the fan for water that condenses and drops down to the basin. These plates are in an embodiment, attached to the PEF and further aid in preventing the formation of biofilms or the buildup of bacteria in the water that is circulating from the building to the water tower. It should be understood that the use of a water tower is not simply limited to its ability to cool buildings but may also be used to cool water in any industrial process including for factories, utility companies, or other uses.

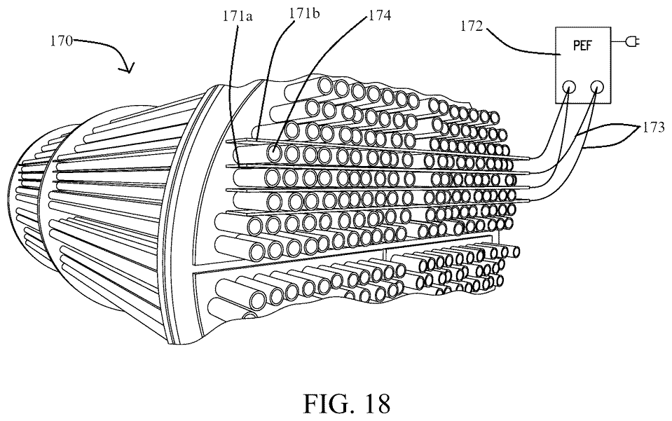

FIG. 18 shows another heat exchanger 170 that can be used to facilitate the cooling of water. Water generally passes through tubes 174 and plates 171a and 171b are positioned to run the entire length of the tubes 174 meaning that biofilm formation and bacterial buildup is precluded throughout the length of the tubes. PEF generator 172 sends pulses through wires 173 to the plates 171a and 171b.

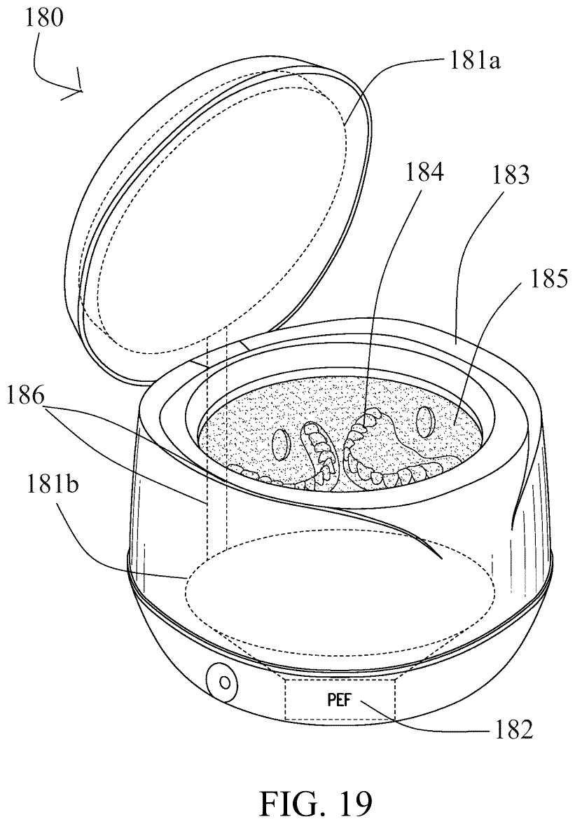

FIG. 19 shows a perspective view of a storage container for dentures or false teeth, or other personal equipment (such as contacts, prostheses, toothbrushes, or fake eyes).

When not in use, dentures and/or retainers are normally stored in a closed container in water or a denture/retainer cleanser solution to keep them moist and retain their shape. Dentures/retainers may be soaked overnight (or during the day) in water or a mild denture-soaking solution. Manufacturer's instructions generally tell the denture wearer to avoid leaving dentures in water for too long, as this can sometimes lead to warping and bacterial growth. Dentures generally should not be disinfected by using hot or boiling water, as this sometimes causes them to warp. Moreover, many solutions that may be used to eliminate bacteria or biofilms are toxic and so should generally be avoided. Thus, the device of the present invention is ideally suited for this purpose.

As shown in FIG. 19, the present pulsed field parameters for the device of the present invention is ideally suited for a situation where biofilms and bacteria can be removed and/or prevented from building up on dentures/retainers by utilizing the device of the present invention. The denture/retainer container 180 comprises a bottom 183 that holds water 185. The PEF generator 182 is shown at a location that is present in the bottom 183. It should be understood that the PEF generator may be at a different location. The PEF generator generates a pulse that is sent via wires 186 to plates 181a and 181b therefore preventing the formation of biofilm and/or bacterial buildup in the water 185. It should be understood that this device can be used in connection with any medical device that may be placed/stored in water (or an aqueous or disinfecting solution) in which the electric field pulses prevent and/or diminish bacterial/biofilm buildup.

In an embodiment, the present invention may use a pulsed electric field that takes any of a plurality of waveforms including but not limited to rectangular, bipolar rectangular, continuous rectangular, exponentially decaying, damped oscillating, triangle wave, sawtooth wave, variable-edge-time pulse, return-to-zero pulses, pulses with jitter, and bursts of arbitrary pulses.

In a variation, the present invention contemplates overlaying or alternating these waveforms. For example, the pulse duration may be between 1 ns to 10 seconds, the pulse rise and fall times may be from 1 ns to 10 seconds, and the pulse frequency may be between 1 μHz to 100 MHz. The present invention contemplates using variable pauses between pulse treatment wherein the delays range from 1 second to 1 day.

In an embodiment, the device to create electric fields may comprise one or more of the following components:

Pulse generator, pattern generator, arbitrary linear waveform generation, function generator, AC high voltage power source, DC high voltage power source, AC to DC converter, voltage converter, voltage amplifier, phase register, phase-to-amplitude converter, digital to analog converter, numerically controlled oscillator, reference clock, phase accumulator, lookup table, pulse transformer, triggering circuit, corona-stabilized switch, thyratron switches, semi-conductor switches, ignitron pulses, tetrode switches, spark gap switches, timing control switches, energy storage capacitor bank, charging current limiting resistor, inductors, resistors, direct digital synthesis generators, phase-locked-loop generators, or digital-to-analog generators, oscilloscope, surge protectors, fuses, circuit breaker, control unit, computer, and/or cooling systems.

In an embodiment, the pulsing parameters for the power supply are between 0.1 kV/cm to 20 kV/cm. or alternatively from 2 and 20 kV/cm and between 1 μHz and 100 MHz, or alternatively between 0.1 Hz and 100 MHZ, or alternatively between 5 kHz and 60 kHz. The electric fields are strong enough to disrupt the cells but are not as high as those used for disinfection. A power source and the frequency ranges is chosen to reduce the cost of the device because the power sources cost less and to make the devices is safe for public use. In an embodiment, the electric field does not need to be applied continuously; the power source is on for a short time and off for a much longer time, therefore reducing the duty cycle to increase efficiency. The choice of a specific duty cycle is dependent on the doubling time for the bacterial species targeted. In a variation, the duty cycle is chosen so as to get an essentially continuous electric field.

The device outlined is innovative in that it uses pulsed electric fields as a way to prevent biofilm growth and bacterial attachment as opposed to disinfection. Previous systems have been made where higher voltage electric fields were used to kill bacteria, but the product of the present invention, in an embodiment, is not designed to kill.

In a preferred embodiment, the present invention is comprised of an electrode surrounded by an insulator to produce an electric field without having the risk of current flowing through the human mouth. In one embodiment, a small neon transformer is powering the invention. In a preferred embodiment, the aforementioned electrode is constructed out of copper. This is because of its low resistance. In another preferred embodiment, the present invention will have a handle that is comprised of a ceramic tube, further insulated by a rubber coating. In another embodiment, the area of the electrode that will be placed in a human mouth employs a spiraling copper wire sealed with medical-grade epoxy, placed between two ceramic pieces, and is then subsequently encapsulated by food-grade rubber. This ensures that little or no current will flow from the handle, or the mouthpiece, to the user. In a highly preferred embodiment, this insulated electrode for removing biofilm from one's mouth will be combined with a standard toothbrush.

In an embodiment, the present invention relates to tubing that comprises one or more electrodes that run a length of the tubing, the one or more electrodes designed and configured to receive one or more pulsed electric fields, the one or more pulsed electric fields set to a strength sufficient to kill bacteria and/or decrease or prevent formation of a biofilm in water or an aqueous composition that passes through the tubing.

In a variation, two electrodes are present. In a variation, three electrodes are present. In a variation, four electrodes are present. In a variation, five electrodes are present. In a variation, more than five electrodes are present.

In a variation, the electrodes (for example, two electrodes) run the length of the tubing, the two electrodes running parallel to each other, the two electrodes never touching each other and positioned at between 90 and 180 degrees from each other on an outer circumference of an inner foil wrap. In a variation, the inner foil wrap runs the length of the tubing, the inner foil wrap forming a cylinder and being positioned on an outer circumference of a plurality of inner tubes that carry the water or the aqueous composition. In a variation, the two electrodes are positioned between the inner foil wrap and insulation, the insulation positioned on an outer surface of the two electrodes and the inner foil wrap. In a variation, an outer surface of the insulation is covered by an outer foil wrap. In a variation, the one or more electrodes are designed and configured to receive the pulsed electric field. In a variation, the tubing is also cooled. In a variation, the strength of the one or more pulsed electric fields is 0.1-20 kV/cm with a frequency range of 1 μHz to 100 MHz.

In an embodiment, the present invention relates to a device that comprises one or more electrodes, and optionally comprises fills, the one or more electrodes designed and configured to receive one or more pulsed electric fields, the device comprising a cooling tower or an ice machine, the one or more pulsed electric fields set to a strength sufficient to kill or prevent growth of bacteria and/or decrease or prevent formation of a biofilm in water or an aqueous composition that passes through the cooling tower, or is present in the ice machine. In a variation, the device is the cooling tower, and the cooling tower further comprises louvres and a drift eliminator. In a variation, the one or more pulsed electric fields are set to the strength of 0.1-20 kV/cm with a frequency range of 1 μHz to 100 MHz.

In an embodiment, the present invention relates to a method of preventing or slowing bacterial growth in a device or preventing or slowing the formation of a biofilm in water or an aqueous solution in or operationally connected to the device, the method comprising: employing at least one electrode in said device, said at least one electrode designed and configured to receive one or more pulsed electric fields from a pulsed electric field generator, said one or more pulsed electric fields of a strength sufficient to prevent or slow bacterial growth in the device or prevent or slow the formation of a biofilm in the water or the aqueous solution.

In a variation, the device comprises a cooling tower, beer dispensing lines, soda dispensing lines, an ice machine, or a container designed and configured to hold dentures and/or retainers. In a variation, the pulsed electric field generator comprises one or more of a pattern generator, an arbitrary linear waveform generation, a function generator, an AC high voltage power source, a DC high voltage power source, an AC to DC converter, a voltage converter, a voltage amplifier, a phase register, a phase-to-amplitude converter, a digital to analog converter, a numerically controlled oscillator, a reference clock, a phase accumulator, a lookup table, a pulse transformer, a triggering circuit, a corona-stabilized switch, thyratron switches, semi-conductor switches, ignitron pulses, tetrode switches, spark gap switches, timing control switches, an energy storage capacitor bank, a charging current limiting resistor, inductors, resistors, direct digital synthesis generators, phase-locked-loop generators, digital-to-analog generators, an oscilloscope, surge protectors, fuses, a circuit breaker, a control unit, a computer, and/or cooling systems. In a variation of the method, the strength of the one or more pulsed electric fields is between 0.1-20 kV/cm with a frequency range of 1 μHz to 100 MHz. In a variation of the method, the strength of the one or more pulsed electric fields is between 5-10 kV/cm with a frequency range of 100 μHz to 10 MHz.

In an embodiment of the present invention, the PEF and the device of the present invention can be used on the hull of a ship with a single electrode. Ships are known to undergo biofouling (biological fouling) which is the accumulation of microorganisms, plants, algae, or small animals where it/they is/are not wanted on surfaces of ships and submarine hulls. Thus, in an embodiment, the device may be positioned so as to work on ship hulls, ballasts, and propellers or alternatively, they may be positioned near docks where there are water inlets, pipework, and/or grates.

In other embodiments of the present invention, the PEF and device of the present invention can be used in liquid storage or handling containers including but not limited to well water retrieval systems, water storage systems, septic tanks, fermentation tanks, water tanks, chemical tanks, hummingbird feeders, and stainless steel reactors. Even in systems with regular cleaning (for example with alkaline, acidic, or sanitation products), a reduction of biofilm on the inner walls or near/around fittings can be beneficial. It is understood that a plurality of electrodes can be used to reduce biofilm formation in these containers. In an embodiment, the electrodes can be single plate electrodes which can independently produce a PEF, on various independent duty cycles, to protect the entire surface of a container. The electrodes can be a variety of sizes, independently only covering a portion of the affected surface. In an embodiment, the invention can use a single electrode.

The many elements of the present invention make it unique in the field. Although this invention has been described with a certain degree of particularity, it is to be understood that the present disclosure has been made only by way of illustration and that numerous changes in the details of construction and arrangement of parts may be resorted to without departing from the spirit and the scope of the invention.

Various other components may be included and called upon for providing for aspects of the teachings herein. For example, additional materials, combinations of materials and/or omission of materials may be used to provide for added embodiments that are within the scope of the teachings herein. In the present application a variety of variables are described, including but not limited to components and conditions. It is to be understood that any combination of any of these variables can define an embodiment of the disclosure. Other combinations of articles, components, conditions, and/or methods can also be specifically selected from among variables listed herein to define other embodiments, as would be apparent to those of ordinary skill in the art.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

While the disclosure refers to exemplary embodiments, it will be understood by those skilled in the art that various changes may be made and equivalents may be substituted for elements thereof without departing from the scope of the disclosure. In addition, many modifications will be appreciated by those skilled in the art to adapt a particular instrument, situation or material to the teachings of the disclosure without departing from the spirit thereof. Moreover, it is contemplated and therefore within the scope of the present invention that any embodiment or feature that is disclosed herein can combined with any other feature as long as those features are not incompatible.

Furthermore, when a range is disclosed herein it is contemplated that any whole number (i.e., any integer) within that range is contemplated as a potential end point for any subgenus. Therefore, it is intended that the disclosure not be limited to the particular embodiments disclosed. Rather, the invention is defined by the following claims.

We claim:

1. Tubing that comprises one or more electrodes that run a length of the tubing, the one or more electrodes designed and configured to receive one or more pulsed electric fields, the one or more pulsed electric fields set to a strength sufficient to kill bacteria and/or decrease or prevent formation of a biofilm in water or an aqueous composition that passes through the tubing.

2. The tubing of claim 1, wherein two electrodes are present.

3. The tubing of claim 2, wherein the two electrodes run the length of the tubing, the two electrodes running parallel to each other, the two electrodes never touching each other and positioned at between 90 and 180 degrees from each other on an outer circumference of an inner foil wrap.

4. The tubing of claim 3, wherein the inner foil wrap runs the length of the tubing, the inner foil wrap forming a cylinder and being positioned on an outer circumference of a plurality of inner tubes that carry the water or the aqueous composition.

5. The tubing of claim 4, wherein the two electrodes are positioned between the inner foil wrap and insulation, the insulation positioned on an outer surface of the two electrodes and the inner foil wrap.

6. The tubing of claim 5, wherein an outer surface of the insulation is covered by an outer foil wrap.

7. The tubing of claim 6, wherein the one or more electrodes are designed and configured to receive the pulsed electric field, and a pulse duration is between 1 ns to 10 seconds, and a pulse rise and fall times are from 1 ns to 10 seconds.

8. The tubing of claim 1, wherein the tubing is also cooled.

9. The tubing of claim 1, wherein the strength of the one or more pulsed electric fields is 0.1-20 kV/cm with a frequency range of 1 μHz to 100 MHz.

10. A device that comprises one or more electrodes, and optionally comprises fills, the one or more electrodes designed and configured to receive one or more pulsed electric fields, the device comprising a cooling tower or an ice machine, the one or more pulsed electric fields set to a strength sufficient to kill or prevent growth of bacteria and/or decrease or prevent formation of a biofilm in water or an aqueous composition that passes through the cooling tower, or is present in the ice machine.

11. The device of claim 10, wherein the device is the cooling tower, and the cooling tower further comprises louvres and a drift eliminator.

12. The device of claim 10, wherein the one or more pulsed electric fields are set to the strength of 0.1-20 kV/cm with a frequency range of 1 μHz to 100 MHz.

13. A method of preventing or slowing bacterial growth in a device or preventing or slowing the formation of a biofilm in water or an aqueous solution in or operationally connected to the device, the method comprising: employing at least one electrode in said device, said at least one electrode designed and configured to receive one or more pulsed electric fields from a pulsed electric field generator, said one or more pulsed electric fields of a strength sufficient to prevent or slow bacterial growth in the device or prevent or slow the formation of a biofilm in the water or the aqueous solution.

14. The method of claim 13, wherein the device comprises a cooling tower, beer dispensing lines, soda dispensing lines, an ice machine, a hull of a ship, a fermentation tank, a water tank, a chemical tank, or a container designed and configured to hold medical equipment, dentures and/or retainers.

15. The method of claim 13, wherein the pulsed electric field generator comprises one or more of a pattern generator, an arbitrary linear waveform generation, a function generator, an AC high voltage power source, a DC high voltage power source, an AC to DC converter, a voltage converter, a voltage amplifier, a phase register, a phase-to-amplitude converter, a digital to analog converter, a numerically controlled oscillator, a reference clock, a phase accumulator, a lookup table, a pulse transformer, a triggering circuit, a corona-stabilized switch, thyratron switches, semi-conductor switches, ignitron pulses, tetrode switches, spark gap switches, timing control switches, an energy storage capacitor bank, a charging current limiting resistor, inductors, resistors, direct digital synthesis generators, phase-locked-loop generators, digital-to-analog generators, an oscilloscope, surge protectors, fuses, a circuit breaker, a control unit, a computer, and/or cooling systems.

16. The method of claim 13, wherein the strength of the one or more pulsed electric fields is between 0.1-20 kV/cm with a frequency range of 1 μHz to 100 MHz.
